(19) United States
(12) Patent Application Publication
(10) Pub. No.: US 2018/0296105 A1
(43) Pub. Date: Oct. 18, 2018

(54) WEARABLE PHYSIOLOGICAL MONITORING AND NOTIFICATION SYSTEM BASED ON REAL-TIME HEART RATE VARIABILITY ANALYSIS

(71) Applicants: Michael Blake, Denver, CO (US); Rodney Kugizaki, Oro Valley, AZ (US)

(72) Inventors: Michael Blake, Denver, CO (US); Rodney Kugizaki, Oro Valley, AZ (US)

(21) Appl. No.: 16/009,876

(22) Filed: Jun. 15, 2018

Related U.S. Application Data
(63) Continuation-in-part of application No. 15/592,566, filed on May 11, 2017, now Pat. No. 10,022,057.
Application No. 15/592,566 is a continuation-in-part of application No. 15/004,345, filed on Jan. 22, 2016, now Pat. No. 9,655,532.
(60) Provisional application No. 62/182,261, filed on Jun. 19, 2015.

(51) Int. Cl. A61B 5/024; A61B 5/00; A61B 5/04; A61B 5/0456; A61B 5/0408

(52) U.S. Cl. CPC A61B 5/02405; A61B 5/746; A61B 5/7282; A61B 5/04012; A61B 2560/0214; A61B 5/04085; A61B 5/6831; A61B 5/0022; A61B 5/0456

(57) ABSTRACT

Methods, devices, and systems for monitoring heart rate variability (HRV) are presented. The HRV monitoring systems and devices are adapted to give immediate feedback to the subject concerning their current condition and any pertinent changes in their condition. The HRV monitoring systems and devices detect, analyze, and assess HRV against a pre-determined application, user needs, against pre-determined limits, or user specific baselines or a combination of both pre-determined limits and user specific baselines. They also have the ability to provide real time notifications based on the system's assessment of a user's heart rate, HRV and changes in the HRV.

### CROSS REFERENCE

This application is a continuation-in-part and claims benefit of U.S. application Ser. No. 15/592,566, filed May 11, 2017, which is a continuation-in-part and claims benefit of U.S. patent application Ser. No. 15/004,345, filed Jan. 22, 2016, now U.S. Pat. No. 9,655,532, which is a non-provisional and claims priority to U.S. Provisional Patent Application No. 62/182,261, filed Jun. 19, 2015, the specification(s) of which is/are incorporated herein in their entirety by reference.

### FIELD OF THE INVENTION

The present invention relates to methods, systems, and devices for real-time monitoring of heart rate variability (HRV), more particularly to HRV monitoring systems and devices that are adapted to give immediate feedback to the subject concerning their current physiological condition and any pertinent changes in their physiology.

### BACKGROUND OF THE INVENTION

Heart Rate Variability (“HRV”) has been widely used as a scientific measurement for monitoring the physiology of both human and animal subjects. HRV is the physiological characteristic of the variation in timing between heartbeats. The heartbeat originates in specialized tissue in the heart called the sino-atrial (“SA”) node, continuously generating an electrical impulse that spreads throughout the heart muscle. This initiates the process of heart muscle contraction, a well-synchronized pump that sequentially constricts all 4 chambers of the heart (two atria and two ventricles.)

The SA node signals (approximately 100-120 impulses per minute when the heart is at rest) are regulated by the autonomic nervous system (“ANS”) by inhibiting some of the electrical impulses. The net effect results in a normal resting heart rate (in healthy individuals) of about 55 to 100 beats per minute (at rest). This autonomic nervous system is the part of the nervous system that is not under conscious control. It controls the organs and systems of the body that are rhythmic, regular, and automatic such as breathing, digestion, and heart rate. There are two branches of the autonomic nervous system: sympathetic and parasympathetic.

The sympathetic nervous system provides the basal heartbeat (“HB”) rhythm based on overall need. This response of the heart rate to normally encountered levels of sympathetic stimulation is modulated by parasympathetic stimulation. This heartbeat response to the parasympathetic nervous system, in contrast to the sympathetic nervous system, occurs rapidly and frequently. The deceleration of the heartbeat is almost instantaneous. It only takes 1 or 2 heartbeats to see these changes take place, slowing the heart rate.

HRV analysis can be used in both clinical and non-clinical applications for a diverse range of evaluations. In healthy individuals, the HR is variable. It fluctuates and, generally, greater variability (or HRV) correlates with better health. Higher HRV indicates a healthy autonomic nervous system, and in particular, healthy balance between the sympathetic and parasympathetic systems. A decreased HRV is an early, accurate indicator that the autonomic nervous system is out of balance. The lower the HRV, the greater the imbalance in autonomic control and the greater the likelihood of poor health, both now and in the future, although, while a lower HRV may indicate a poorer health state, there can be exceptions. Such exceptions are often related to normal response mechanisms including, but not limited to stress, intense physical activity, and fatigue.

Clinical applications for HRV analysis are related to cardiac health, and are indications that are shown to directly relate to health changes with many chronic and critical health conditions. Included are, but not limited to, risk of a cardiac event, occurrence of diabetes, episodic and chronic mental health conditions, sleep apnea, SIDS, exposure to and incidence of allergic reactions.

In non-clinical applications, it has been shown that HRV is effective in indicating a variety of physiological conditions. During vigorous exercise, HRV has been shown to be a marker for entering lactate threshold or anaerobic metabolism. Further, it is shown to be an indicator of physical fatigue, exercise capacity, endurance, and overall fitness. Application has been found to be useful in assessing physiological-behavioral conditions, such as stress in trainee stock market traders and driver fatigue.

There are several ways to measure and analyze HRV. Heart rate signals are obtained through electrocardiogram (“ECG”) or by pulse wave measurement called “Photoplethysmography” (“PPG”). The most accurate clinical determination of HRV is derived from measuring the duration of the intervals between contractions of the heart, called interbeat intervals, on ECG (or EKG). In contrast, PPG is less invasive, simpler to apply, and can conveniently access capillaries in a fingertip or the earlobe. Using differential light absorption characteristics and an optical sensor, PPG detects changes in the pulse waves generated by blood flow through the microcirculation. In this way an accurate estimate of HRV can be obtained.

The present invention features a device and method for real-time HRV monitoring. The HRV systems and devices of the present invention are adapted to give immediate feedback to the subject concerning their current physiological condition and any pertinent changes in their physiology.

A few studies that outline some applications of HRV benefiting from real-time feedback include, but are not limited to, clinical applications with real time relevance such as anticipation of mood changes in patients with Bipolar Disorder, alerting the onset of infant physiological dysfunction during sleep, early warning of epileptic seizure, food allergy alerting, and sleep apnea; and non-clinical applications with real time relevance such as predicting the onset of lactate threshold in endurance athletes, warning of physiological effects of pollution, particularly volatile organic compounds (“VOCs”), alerting the onset of driver fatigue, and monitoring professionals in high stress occupations (e.g., air traffic controllers). These scenarios and many others may benefit from the accurate monitoring, analysis and real time alerting, to a relevant change in physiology as indicated by a change in HRV.

### SUMMARY OF THE INVENTION

The present invention features a device providing determination, analysis, and feedback of HRV data to users on a real-time basis. In addition to the detection, analysis, and assessment of HRV data against a pre-determined application or user need, it has the ability to provide real time notifications based on the system's assessment of a user's heart rate variability and, in particular, changes in heart rate variability that may be pertinent to a specific application of this invention. This invention may further help a user to take appropriate action based on his/her specific needs.

One aspect of this invention is to provide monitoring and assessment of the impact of HRV when incorporated with other relevant factors, historical baselines, and temporal changes. Further, such temporal changes, of either or both HRV and other factors, are assessed for the providing of significant indication of occurring or impending clinical or non-clinical conditions for a user.

According to one embodiment, the invention features a wearable device for real-time detection, analysis, and application of heart rate variability (HRV). The device may comprise a chest strap integrated with one or more strap electrodes to detect a plurality of consecutive QRS waves and a battery-powered and self-contained processing circuit. The circuit may comprise a microprocessor configured to receive at least one additional input unrelated to the QRS waves detected by the strap electrodes, an ECG analog front end circuit coupled between the microprocessor and one or more one strap electrodes to provide a signal gain control to a strap electrode output such that the microprocessor receives signals with a desired amplitude, a non-volatile memory storing computer-readable instructions, and a notification means to receive a real-time notification to generate a user alert. The notification means may be a haptic indicator, an audio indicator or a visual indicator. When the computer-readable instructions are executed by the microprocessor, the microprocessor can perform operations comprising detecting a peak, a rising edge or a declining edge of an R-wave from each of the QRS waves detected; triggering an interrupt when the peak, the rising edge, or the declining edge crosses a trigger point, where the trigger point is a predetermined or dynamically adjusted value; recording a time value each time the interrupt is triggered; determining an R-R interval each time the time value is recorded; generating a temporal R-R interval array, the R-R interval array being stored within the memory and comprising a plurality of determined R-R intervals and corresponding time values; generating an HRV measurement based on the temporal R-R interval array; and comparing the determined HRV measurement to an HRV threshold and outputting a real-time notification when the HRV threshold is reached. The HRV threshold can be determined at least by the additional input unrelated to the QRS waves.

Not wishing to limit the present invention, temporal interval arrays may comprise of, but is not limited to, unique intervals other than R-R interval arrays, such as the P wave interval, PR interval, QRS interval, ST interval, or QT interval. Further, arrays may also comprise additional parameters of the ECG signal, including specific wave characterization (e.g., p-wave, t-wave) as well as non-wave characterization (e.g., S-T segment depression or elevation).

While there are many configurations and implementations of HRV analytical systems, this invention provides unique improvements and capabilities offering significant advantages over existing systems. One such improvement is the feature of employing an analog voltage comparator to detect specified characteristics of the QRS wave. This feature is critical because, traditionally, the QRS wave is sampled by an analog to digital converter (“ADC”) disposed within the microcontroller and peak detection algorithms are executed by the microcontroller to determine the duration of the R-R interval, as can be seen in Scott (2010/0274308) and Kaiser (2007/0021815). This traditional approach requires additional signal processing to obtain sufficient resolution and accuracy for determining the specific heartbeat timings and, consequently, the duration of R-R intervals. The approach of the present invention provides a marked improvement over existing methods and accounts for greater capture efficacy and more responsive adaptation to unusual circumstances (e.g. a leads off situation), while maintaining the integrity of the data set used for calculation of the HRV values. By virtue of the stability provided by the device disclosed herein, it is thus sometimes unnecessary to provide for additional signal analysis and correction of the basic QRS complex waveform exclusively for the purposes of determining the R-R timing.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### Definitions

As used herein, the trigger point is the voltage level at which the R-wave triggers an interrupt.

As used herein, the HRV threshold is the level the selected HRV measurement crosses in order to generate an alert.

As used herein, the R-R interval is the time between two consecutive R-waves (usually expressed in milliseconds).

### BRIEF DESCRIPTION OF THE DRAWINGS

FIG. 1 shows an example of a wearable HRV monitoring device (e.g., compact, integrated, portable, battery powered, etc.).

FIG. 2 shows a detailed block diagram of the front-end detection in a HRV device of the present invention.

FIGS. 3A-3D are a table showing exemplary HRV measurements and associated sample applications involving each specific measurement. Each one of these calculations yields a single measurement. In steady state conditions, these measurements remain fairly constant, but can change with changes in the physiology of the subject. For example, if there is a rapid increase in the value of HF preceding an epileptic seizure, or during exercise, the subject crosses his or her lactate threshold as the value of SD1 drops below 3.0.

FIGS. 4A-4C are a logic flow diagram for an embodiment of the method of the present invention. This embodiment provides for the determination of parameters potentially significant for cardiac arrhythmia based on heart rate and HRV determination.

FIGS. 5A-5C are a logic flow for an alternate embodiment of the method of the present invention. This embodiment provides for the determination of parameters potentially significant for cardiac arrhythmia along with heart rate and HRV determination.

FIGS. 6A-6B is a table showing exemplary ECG Characterization Relationships to Cardiac Arrhythmia (characteristics and measurements). Each one of these characteristics yields a single determination. Under stable health conditions, establishing a user baseline for appropriate characteristics allows for the real time determination of changes in a characteristic, such change can be expressed as a figure of merit for the specific characteristic. These changes and resulting figures of merit may be highly indicative of a change in cardiac condition, especially when combined with other factors. When combined with HRV and other determining factors, this indication may indicate the early onset of a significant cardiac event.

FIG. 7 shows a typical, normal ECG waveform with call outs for the various referenced intervals, segments and waveforms.

FIG. 8 shows an example of an embodiment of this invention, describing a multiplicity of real time notification paths.

One skilled in the art will recognize that various implementations and embodiments of the invention may be practiced in accordance with the specification. All of these implementations and embodiments are intended to be included within the scope of the invention.

### DETAILED DESCRIPTION OF THE INVENTION

In the following description, for the purpose of explanation, specific details are set forth in order to provide an understanding of the present invention. The present invention may be practiced without some or all of these details. The embodiments of the present invention described below may be incorporated into a number of different electrical components, circuits, devices, and systems. Structures and devices shown in schematic diagrams are illustrative of exemplary embodiments and are not to be used as a pretext by which to obscure broad teachings of the present invention.

When the specification makes reference to “one embodiment” or to “an embodiment”, it is intended to mean that a particular feature, structure, characteristic, or function described in connection with the embodiment being discussed is included in at least one contemplated embodiment of the present invention. Thus, the appearance of the phrase, “in one embodiment,” in different places in the specification does not constitute a plurality of references to a single embodiment of the present invention.

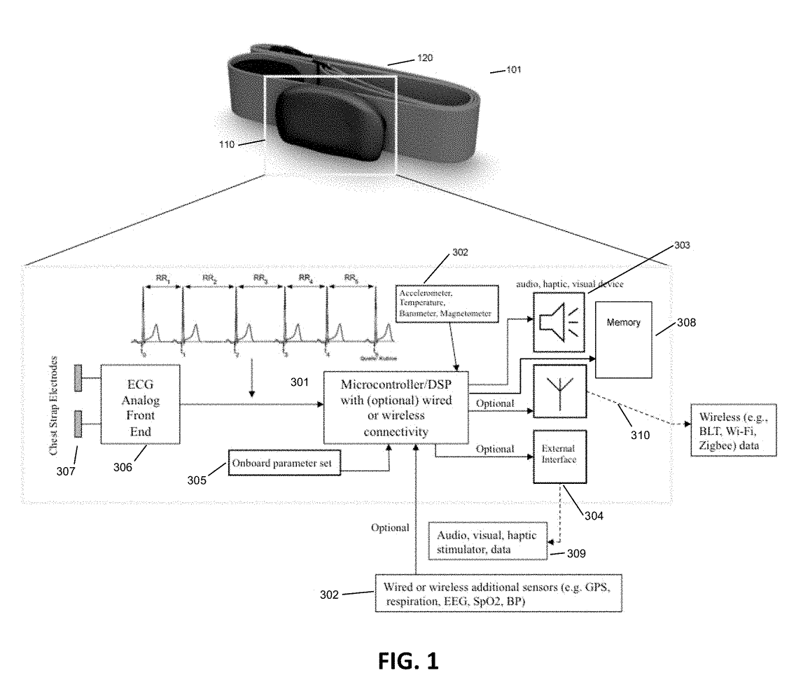

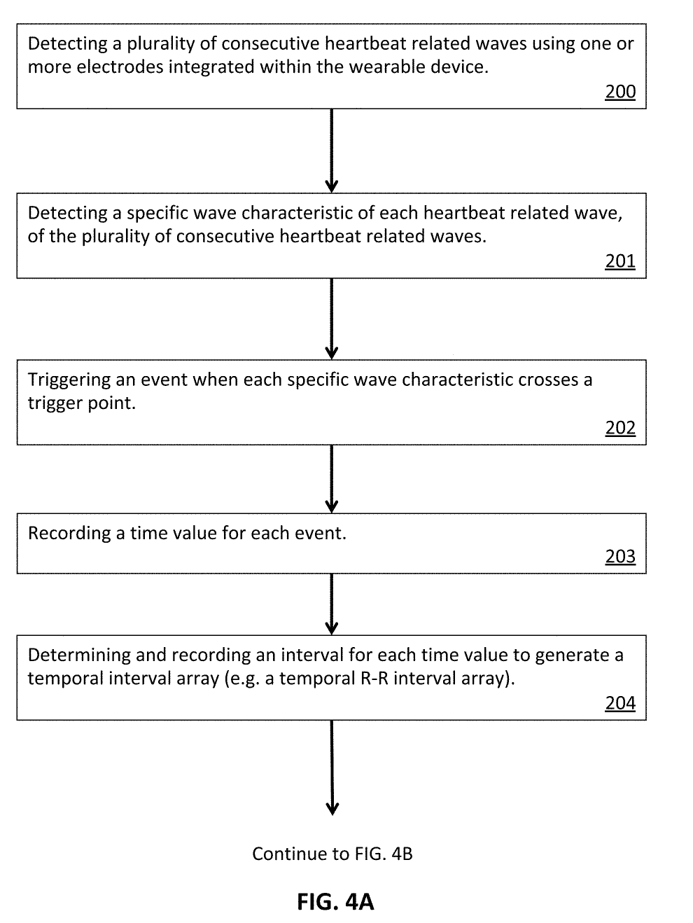

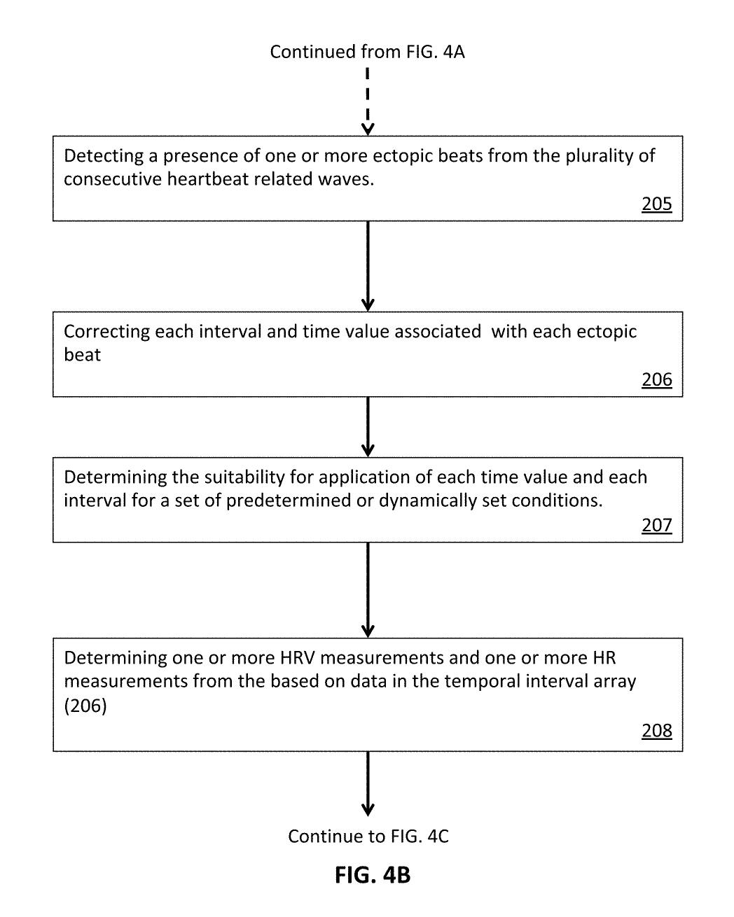

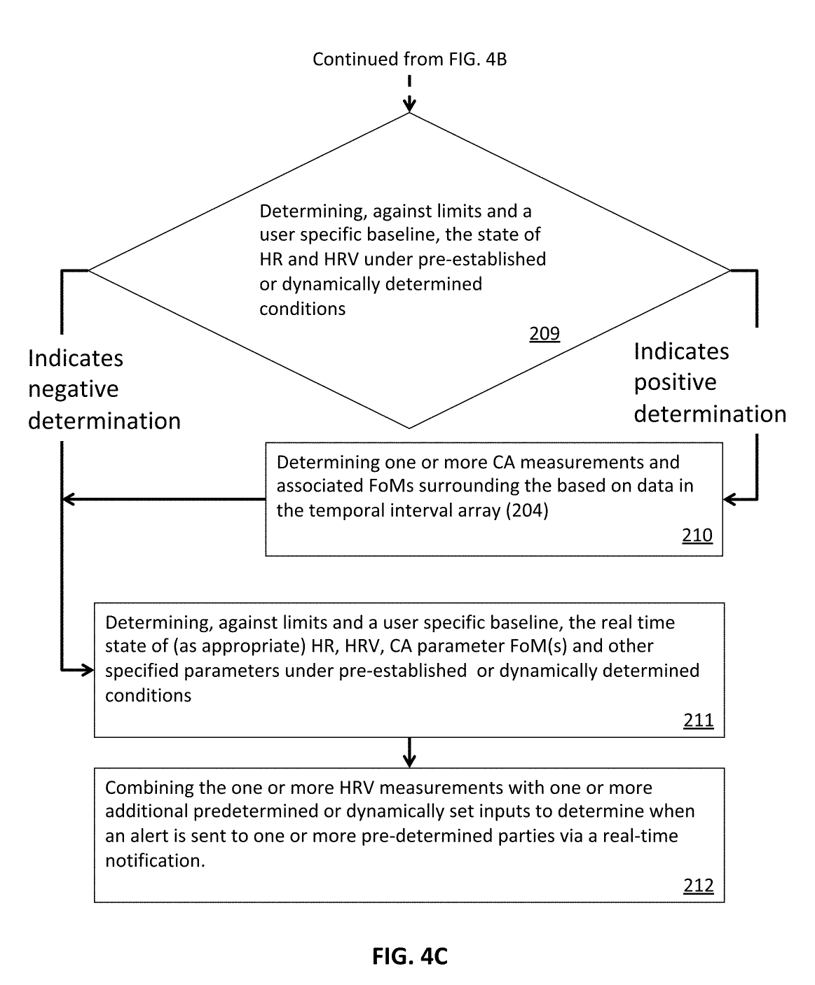

The present invention features a wearable device (101) for real-time detection, analysis, and application of heart rate variability (HRV). Referring now to FIGS. 1-7, in some embodiments the device (101) is compact, battery-powered, wearable, and portable. Not wishing to limit the present invention, the device may comprise of a chest strap (120) and a battery-powered, self-contained, processing circuit (110). The processing circuit (110) may comprise a memory (308) coupled to a microprocessor (301). In one embodiment, one or more strap electrodes (307) may be integrated into the chest strap (120) and configured to detect a plurality of consecutive QRS waves. In alternate embodiments, the microprocessor (301) may be a microcontroller, a digital signal processing (DSP) circuit, a programmable logic circuit, a CPU, etc. The memory (308) may be a non-volatile repository storing computer readable logic/control codes (or instructions) and computer generated data. In some embodiments, the microprocessor (301) and the memory (308) are integrated into one integrated circuit.

In some embodiments, the microprocessor (301) may be a highly integrated, processing and memory unit, while in other embodiments, the microprocessor may be a combination of one or more of a microcontroller, a microprocessor, a digital signal processor, a field programmable gate array, a mixed signal integrated circuit or a system on a chip integrated circuit. The embodiments herein described are meant as exemplary models and the invention is not limited to the examples provided. One skilled in the art will recognize that, depending upon specific application requirements; the embodiment of the microprocessor (301) shall be determined to optimize the performance, size, power consumption and capabilities of the invention.

In further embodiments, the processing circuit (110) may comprise an electrocardiogram (“ECG”) analog front end (“AFE”) integrated circuit (306) coupled between the microprocessor (301) and the one or more strap electrodes (307). The AFE circuit (306) may be configured to provide a necessary signal gain control to one or more output signals emerging from the one or more strap electrodes (307) so that said signals exhibit a desired amplitude before transmission to the microprocessor (301). In additional embodiments, the microprocessor (301) may execute computer-readable instructions stored in the non-volatile memory (308), causing the microprocessor (301) to perform operations such as detecting the rising edge, the peak, and/or the declining edge of an R-wave from each QRS wave. An event may be triggered to capture and recorded a time value when the rising edge, the declining edge, and/or the peak of each R-wave crosses a trigger point. In one embodiment, the trigger point may be a predetermined value or a dynamically adjusted value. In another embodiment, the event may be a time capture, a real-time clock value capture, or an independent timer value capture. Further, an R-R interval may be determined and recorded for each time value to generate a temporal R-R interval array, which is stored in the non-volatile memory (308).

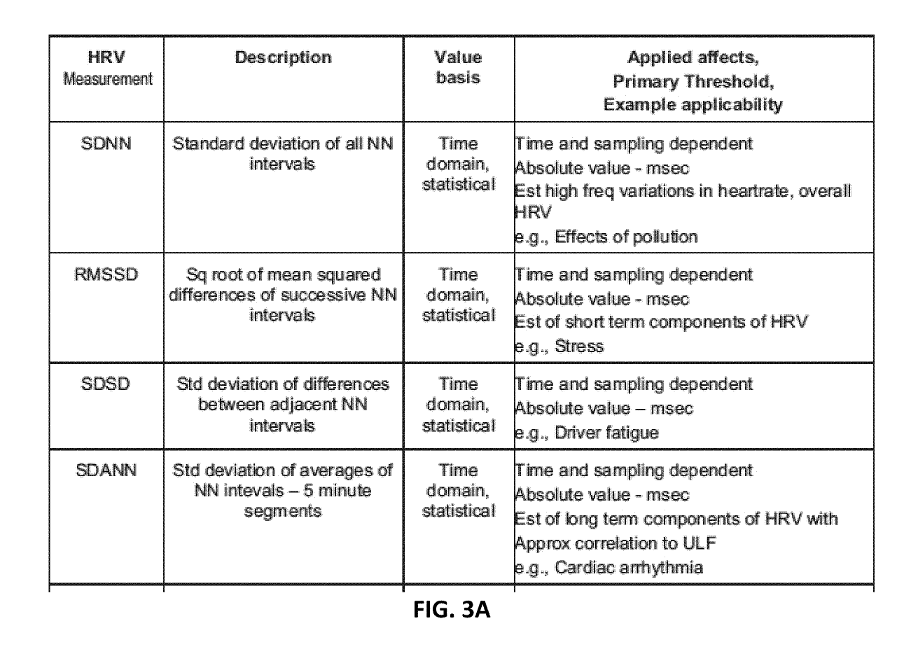

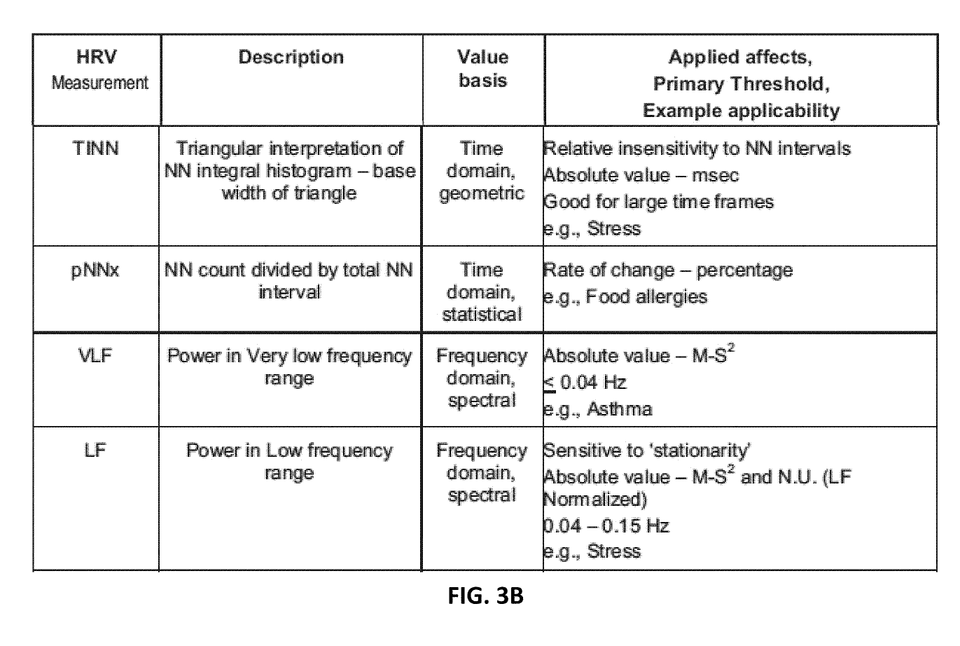

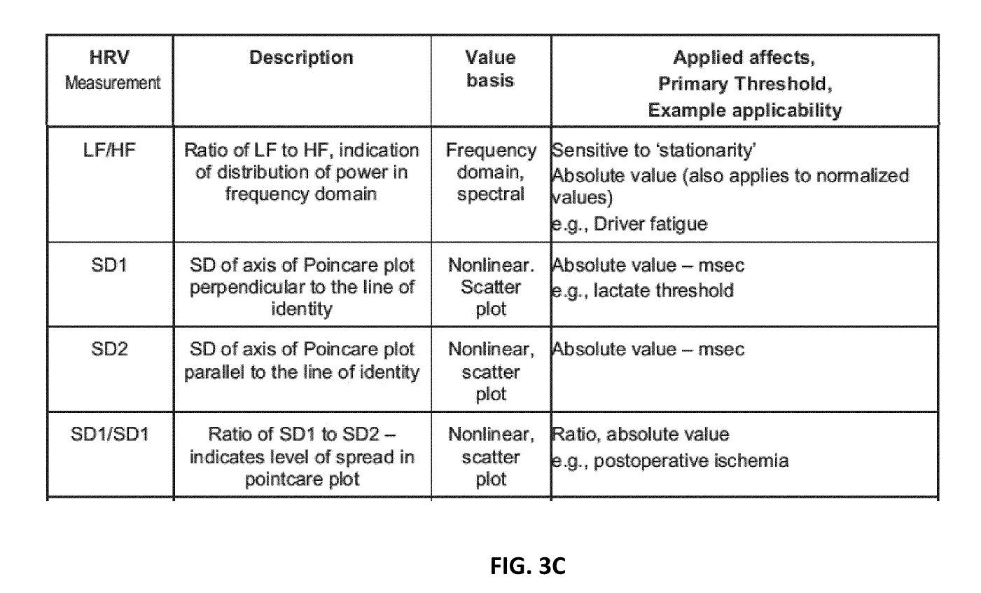

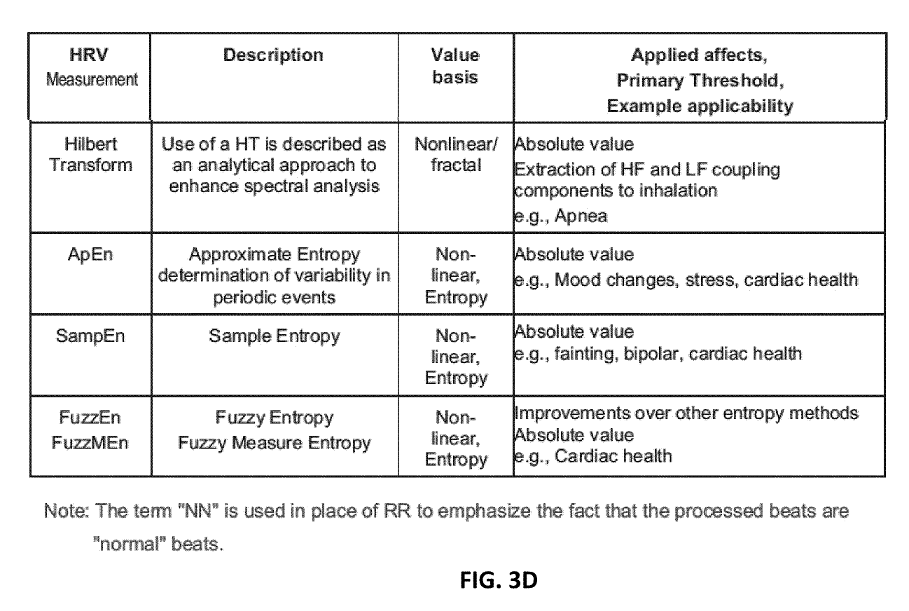

The plurality of consecutive heart beat related waves, each time value, and each interval may then be analyzed for suitability of use for HRV related applications. To accomplish this analysis, additional operations executed by the microprocessor (301) may include determining a presence of one or more ectopic beats from the plurality of consecutive QRS waves and correcting the R-R interval and time value associated with each ectopic beat to produce corrected R-R intervals and time values. Correction may be accomplished by comparing an R-R interval and time value of an ectopic beat to a predetermined value and replacing the R-R interval and time value with a calculated R-R interval value and a calculated time value if the R-R interval and time value exceed the predetermined value. One or more HRV measurements may then be determined based on corrected R-R intervals and time values. Non-limiting examples of methods for generating the calculated R-R interval values and the calculated time values comprise: standard deviation of normal to normal R-R intervals (“SDNN”), root mean square of successive N-N interval differences (“RMSSD”), standard deviation of successive differences (“SDSD”), standard deviation of averages of N-N intervals (“SDANN”), triangular interpolation of N-N interval histogram (“TINN”), NN count divided by total NN interval (“pNNx”), Hilbert Transform, approximate entropy (“ApEn”), sample entropy (“SampEn”), fuzzy entropy (“FuzzEn”), fuzzy measure entropy (“FuzzMEn”), or spectral HRV measurements including: power in very low frequency (“VLF”), power in low frequency (“LF”), power in high frequency (“HF”), a ratio of LF to HF, standard deviation of Poincare plot perpendicular to a line of identity (“SD1”), standard deviation of axis of Poincare plot parallel to the line of identity (“SD2”), or a ratio of SD1 to SD2 (see FIGS. 3A-3D).

In addition to or along with the HRV determination, the ECG waveform, and specifically the PQRST complex, may be analyzed for specific characteristics. Each analysis is temporarily associated with an R value, allowing for an array of values to be determined for characteristics of interest. Non-limiting examples of characteristics include P-wave presence and duration, Pr-R interval duration, QRS interval duration, ST segment duration, ST segment elevation or depression, ST interval duration and the QT/QTc interval duration.

In supplementary embodiments, the one or more HRV measurements are combined with one or more additional predetermined or dynamically set input to determine when an alert is sent to one or more pre-determined parties via a real-time notification. These predetermined or dynamically set inputs may comprise: respiration rate, blood pressure value, body temperature, level of physical motion, heart rate, cardiac arrhythmia characteristics, a pre-determined condition marker (e.g. a blood sugar level), a pre-existing condition specification (e.g., cardiac arrhythmia, diabetes, apnea, epilepsy), or a level of electrodermal activity. In some embodiments, the pre-existing condition specification may comprise: sleep apnea, physical exertion, cardiac arrhythmia, tendency for epileptic seizure, diabetes, or stress. The real time notification may be a haptic indicator, an auditory indicator, or a visual indicator disposed on the device (303). Further, the real time notification may as well be a haptic indicator, an auditory indicator or a visual indicator located off of the device (309) and connected via an external interface (304). An optional wireless interface (310) can further transfer data between the device (101) and an external receiver.

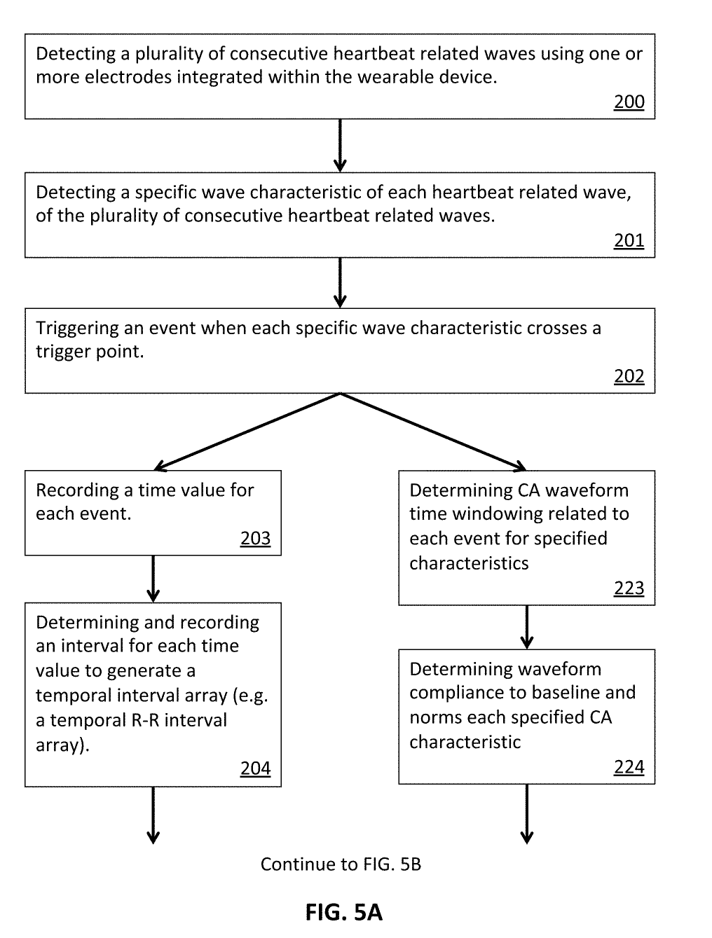

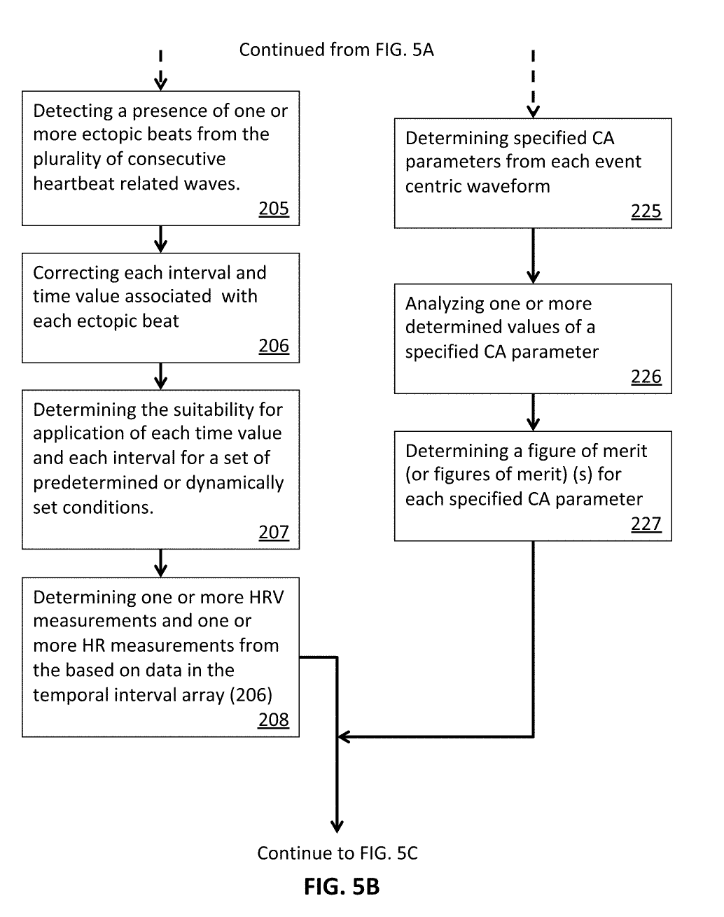

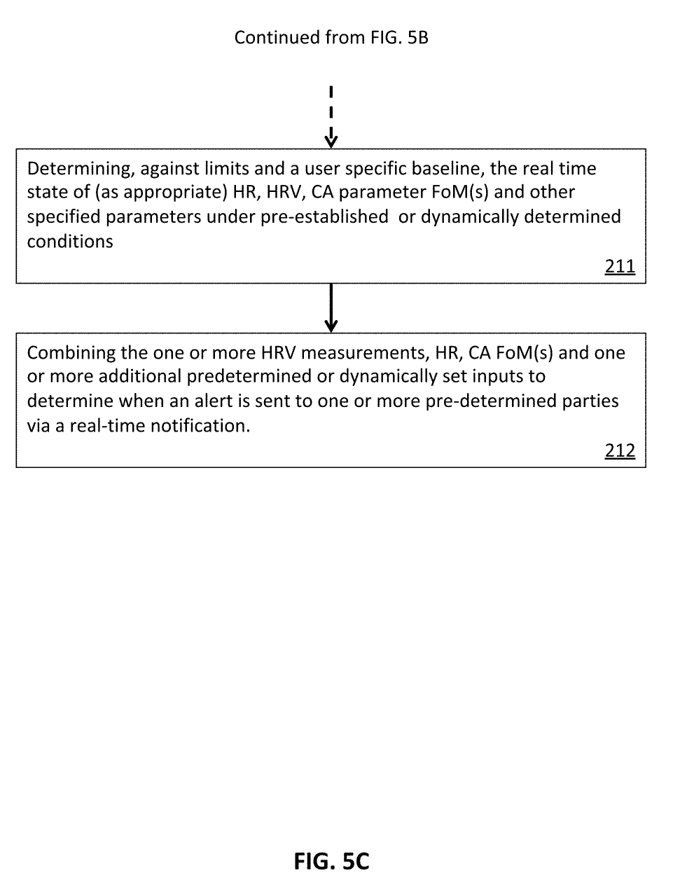

In said embodiments, the one or more HRV measurements are combined with heart rate, one or more inputs specific to the detection of cardiac arrhythmia (FIG. 5) as related to sudden cardiac arrest and conditions specific to the user. While there are several heart conditions that can lead to sudden cardiac arrest (including but not limited to coronary artery disease, enlarged heart, heart attack, valvular heart disease, congenital heart disease, electrical problems in the heart), such conditions can result in cardiac arrhythmia and resulting sudden cardiac arrest. Pre-existing condition specifications, specifically a tendency to cardiac arrhythmia, family history, personal habits, high blood pressure, high cholesterol, obesity, diabetes, or a sedentary lifestyle, are combined with the measurement of HRV, one or more inputs to assess relevant physiological changes relating to cardiac arrhythmia and compared to patient historical baselines (for such physiological changes) to identify the onset of potentially significant cardiac arrhythmia. Such inputs to relevant physiological changes include, but are not limited to ECG analysis factors, including R-R interval, HRV, P-wave determination, PR interval determination, QRS complex interval determination, ST segment/interval determination, ST segment elevation or depression, and QT/QTc interval determination. One or any of these determinations can be compared to baseline and normative values to provide a figure of merit for the factor. The analysis of HRV, HR, and one or more ECG factor can provide a real time determination of suspected impending cardiac events, such a determination with a real time notification can alert a user of the potential of a life-threatening event.

In additional embodiments, the processing circuit (110) may also comprise an onboard threshold set (potentiometer, adjusting switch or digital control value) (305) coupled to the microprocessor (301). The onboard threshold set (305) may receive user inputs for the microprocessor (301) to determine HRV measurements accordingly. The user inputs may be an exercise status input, a health status input, etc. The processing circuit (110) may further comprise an optional connector (304) coupled to the microprocessor (301), which provides the real time notification to the user through the connector (304) and an external earpiece (audio), display or light (visual) or vibratory device (haptic) coupled to the connector (304).

Although the wearable device (100) is shown in FIG. 1 with a chest strap (120), it is understood that other variations may be applicable to the device as well. Such variations could be in the form of, but not limited to, a watch, self-adhesive patch, armband, bra, belt, shirt, pants, earrings, socks, shoes, headphones, headsets, etc. The strap electrodes (307) may be integrated into the chest strap (120) such that the electrodes may be disposed at the right position when the device is worn by the user. In other embodiments, the electrodes could be integrated into multiple devices worn or held by the user.

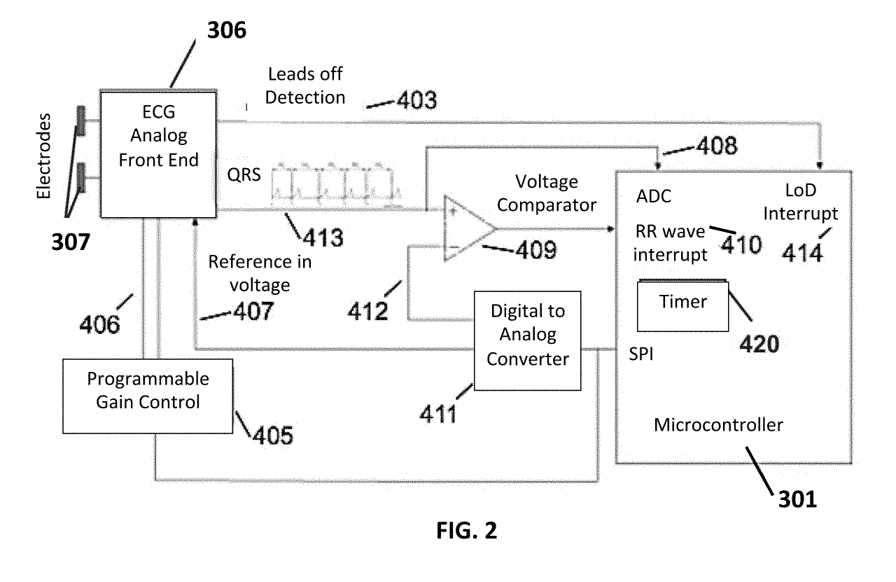

FIG. 2 shows a detail schematic view of the front-end detection in an embodiment of the present invention. Two chest strap electrodes (307) are coupled to the AFE circuit (306). A non-limiting example of the AFE circuit is an Analog chip AD8232. In some embodiments, the AFE circuit (306) contains an internal amplifier to output a conditioned QRS signal (413) comprising multiple QRS complexes to a voltage comparator (409). In other embodiments, the AFE circuit (306) is configured to output a Leads off Detection (LoD) signal (403) when one or both of the chest strap electrodes (307) have been disconnected from the user, thereby compromising the integrity of the ECG data. The LoD signal (403) may be coupled to a LoD interrupt input pin (414) of a microcontroller (301). The microcontroller (301) can then activate a ‘Fast Restore’ feature of the AFE circuit (306) in order to reset the internal filters so that the AFE circuit (306) is reconfigured to immediately detect a new R-wave. The microcontroller (301) may also use this information to identify, reject, or correct the resulting erroneous R-R interval data. Additionally, an internal timer (420) in the microprocessor (301) can monitor the LoD (403) and in the event that the leads are off longer than a threshold (a long period of time, such as 15 seconds), enable the microcontroller (301) to enter a low power sleep mode, assuming that the user has removed the device.

In some embodiments, in order to optimize the performance of the AFE circuit (306), the microcontroller (301) may adjust the output gain of the AFE circuit (306) to minimize saturation via a programmable gain control (405). Additionally, the microcontroller (301) can adjust the reference level of the AFE circuit instrumentation amplifier (407) in order to optimize the average level of the R pulse baseline. This can be achieved by monitoring and averaging the QRS signal (408) using an ADC and adjusting the reference-in voltage (407) to maintain a very stable baseline.

Traditionally, the QRS signal is sampled by an analog to digital converter within the microcontroller and peak detection algorithms are executed in the microcontroller to determine the duration of the R-R interval. This approach requires signal processing to obtain sufficient resolution and accuracy in determining the specific heartbeat timings and consequently duration of the R-R intervals.

It is an object of this invention, in some embodiments, to employ an analog voltage comparator (409) to detect a specific desired wave characteristic and use that detection event to directly trigger an interrupt (410) on the signal processor. The specific wave characteristic may be, but is not limited to, the steep rising edge of the R-wave, the peak of the R-wave, or the steep declining edge of the R-wave complex. Each may be used to directly trigger an interrupt on the microcontroller, with each embodiment having its own characteristics. Combinations of specific wave characteristics are as well possible and it is not meant to, within this invention, to limit the use of and the combinations of wave characteristics for this detection.

It is an object of this invention, in some embodiments, to use HR and HRV determination as criteria for determining cardiac arrhythmia characteristics (209). Depending upon this determination, cardiac arrhythmia characteristics are determined (210). Heart rate, HRV and CA figure(s) of merit (FoMs) associated with each event of interest can be determined and evaluated against baselines and limits (211). As appropriate, heart rate, HRV and cardiac arrhythmia are combined with other inputs to determine an alert status (212) with a real time alert delivered as necessary.

In other embodiments, as HRV events (202) are determined, the ECG waveform associated with the event is evaluated as it applies to cardiac arrhythmia (223). Specific waveform characteristics are examined against baseline and normal characterization (224) and specified characteristics are determined, analyzed and a figure of merit (FoM) is determined (225, 226, 227). Heart rate, HRV and CA figure(s) of merit (FoMs) associated with each event of interest can be determined and evaluated against baselines and limits (211). As appropriate, heart rate, HRV and cardiac arrhythmia are combined with other inputs to determine an alert status (212) with a real time alert delivered as necessary.

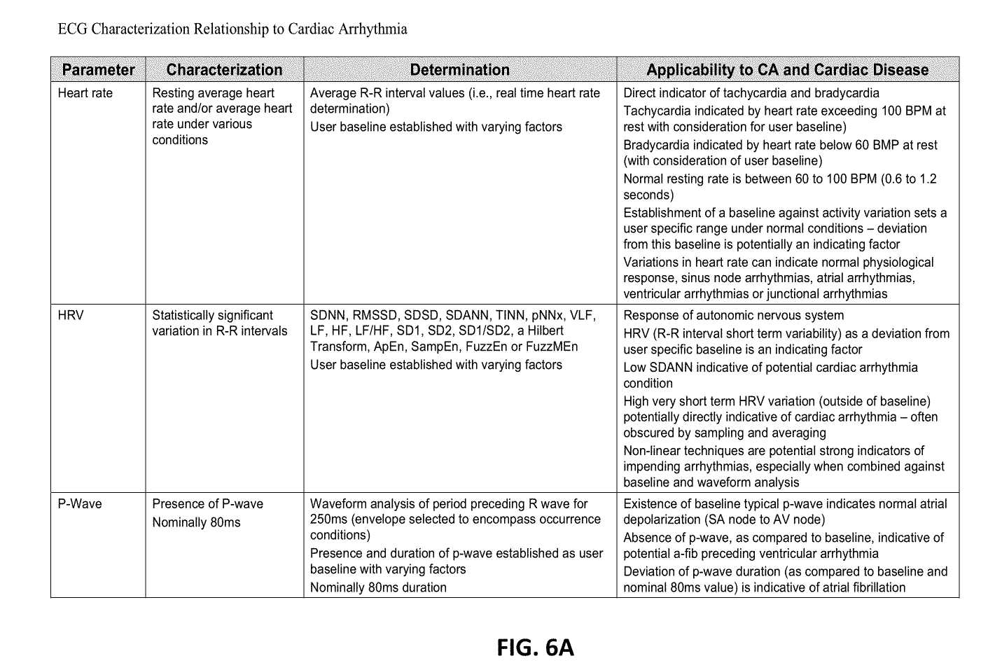

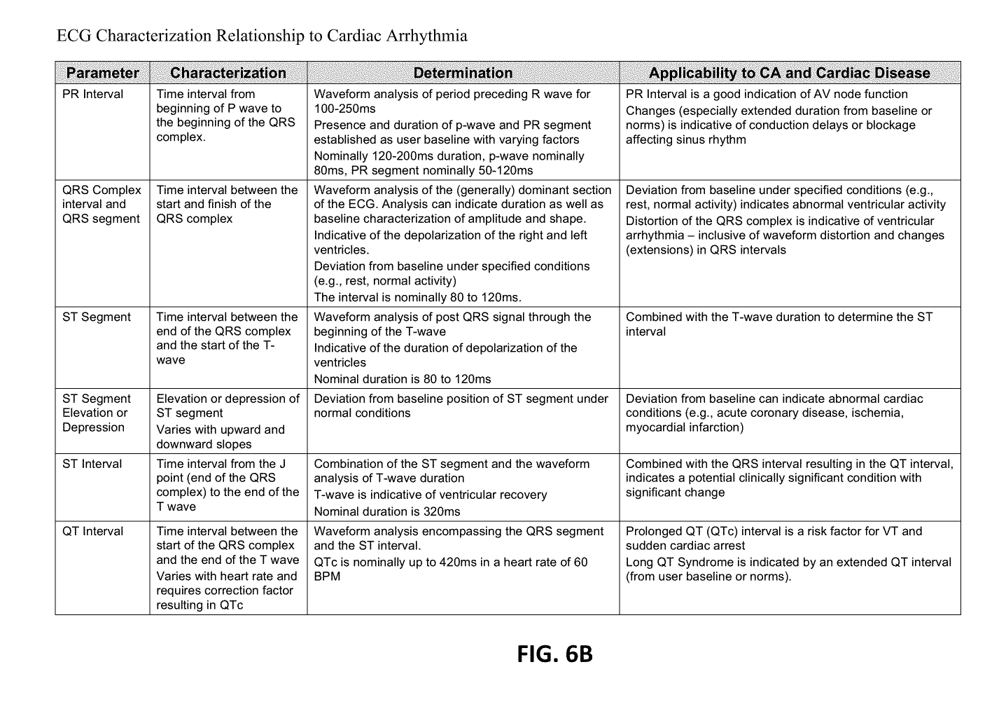

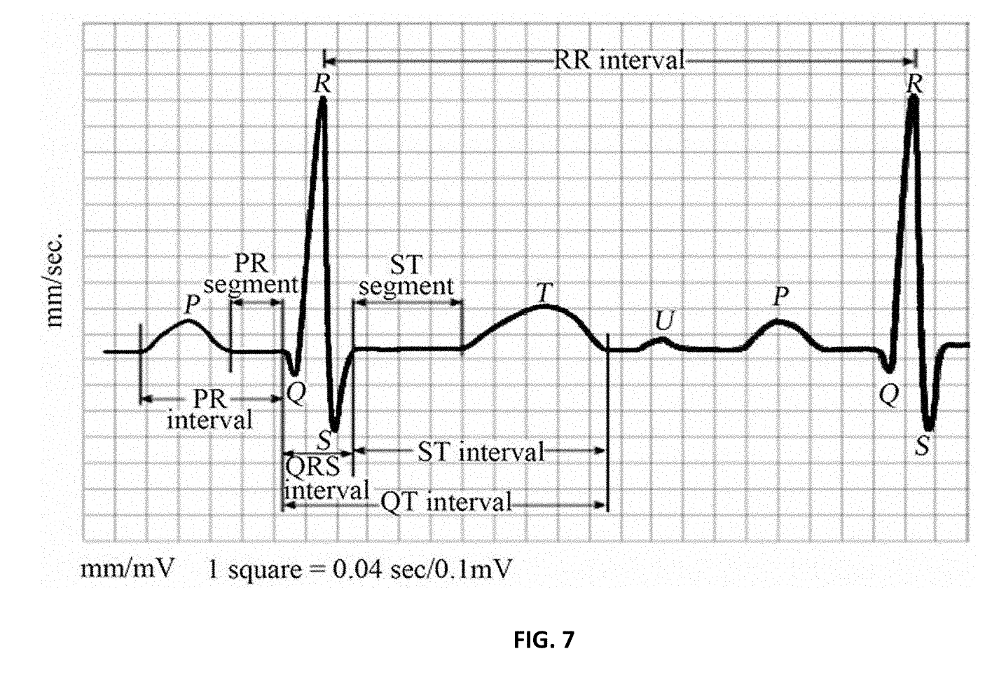

FIGS. 6 and 7 shows that, in some embodiments, the specific wave characteristic may be focused on other characteristics of the ECG output, such as the P-wave, PR interval, QRS complex, QRS interval, QRS segment, ST segment, ST interval, QT interval or J point, including partial selections, upward and download sloping, segment elevations or depressions, and unique groupings of specific wave characteristics thereof. In other embodiments, the specific wave characteristics or threshold intervals arrays may be mathematically manipulated to be a derivative, fraction, or other function of the original value prior to characterization, detection, analysis, or alerting.

A digital to analog converter (“DAC”) (411) connected to the microcontroller may be used to actively set (or dynamically adjust) the threshold (412) based on the magnitude variation of the R-wave between different users or historic data stored within the memory (308). The threshold (412) is used to compare to the conditioned QRS signal (413) by voltage comparator (409). When the conditioned QRS signal (413) reaches the threshold (412), the voltage comparator (409) outputs an interrupt signal (410) to the microcontroller (301). While FIG. 2 shows discrete external components, this is not meant to limit a desired embodiment, as this capability may be discrete or integrated into the microcontroller. It is herein provided for greater clarity of explanation.

The interrupt (410) is used by the microcontroller (301) to obtain a time value from a timer (420) that was reset on the last occurrence of a valid interrupt. The obtained time value is then the most recent R-R interval. This direct and temporal determination of the R-R interval for HRV provides a highly accurate determination of the R-R interval while lowering power requirements, computational requirements, memory storage and timing limitations for computationally intense digitizing/analysis methods. The obtained R-R interval array is stored within the memory (308) for further analysis and history data output when applicable. The precision and accuracy of this determination is only limited by the timing capabilities of the microcontroller (301) and is often provided with sub-millisecond resolution and accuracy.

By virtue of the stability provided by the above described technique, it is sometimes unnecessary to provide for additional signal analysis and correction of the basic QRS complex waveform exclusively for the purposes of determining the R-R timing. Even with this stability, there are advantages to enhanced error detection, improved noise rejection and verification of the specific wave characteristics expected. It is a further object of this invention to enable enhancements in these validations of the base R-R interval determination.

This additional analysis is valuable to provide for the confirmation of viable heartbeat (HB) and HRV data and to determine consistency and viability of the assessment.

As used herein, the term “about” refers to plus or minus 10% of the referenced number.

As is described in the preferred and supplemental embodiments of this invention, real time notification is essential to provide the user with information that can potentially be used to address significant events or episodes at an early, therapeutically advantageous time preceeding the presentation of other symptoms. This real time notification can be delivered via a variety of means, including but not limited to haptic, visual, aural and messaged stimulation. Further, since the information may be indicative of a significant health event or episode, the invention allows for the simultaneous, real time notification of the user, real time notification of a personal health care team (a user's family or other personal relation directly involved in the user's health care), and the real time notification of a health care provider (a physician, health care provider or caregiver) or any combination of these.

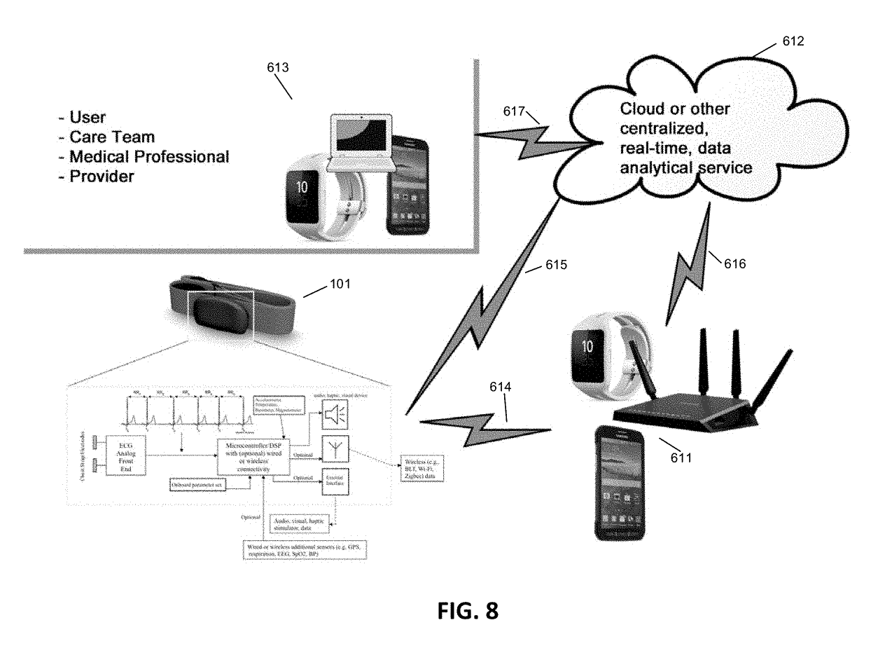

The above described real time notification capability is as described herein and as depicted in FIG. 8 herein. In some instances, the real time path may be directly to the user via a worn device (101). While this is a preferred embodiment of this invention, in other instances, in addition to (or as a replacement of) the worn device's notification, the invention may provide real time notification via wireless communication (614, 615) to a device or a network (611) or a cloud computing system, or a centralized computing system, or a data analytical service (612), with notification delivered (617) via this capability through a computer, smart phone, smart watch or other data connected device (613) or notification delivered through a computer, smart phone, smart watch or other data connected (614) device (611) without utilizing a cloud computing system, or a centralized computing system, or a data analytical service (612).

As is described in the preferred and supplemental embodiments of this invention, real time notification, taking the form of multiple, tiered notifications, is intended. This multiple, tiered, real time notification enables the notification at the earliest indication of potential events or episodes and supports subsequent detection, analysis and further real time notifications to optimize the interaction with a user and/or their care team. A first notification may inform the user, and with training, specific instruction and/or specific messaging, may cause the user to change behavior, enabling further and/or modified detection, analysis and real time notification with modified conditions facilitating the reduction or elimination of false positive or negative analysis and enhanced detection, analysis and notification. Such improvement enhances the efficacy of the invention.

In example, a person known to have propensity for cardiac arrhythmia monitors routinely for heart rate and HRV using the device as described herein. The monitor is, per the described invention, capable of determining heart rate, HRV, activity (through motion monitoring) and can derive respiration from the ECG waveform.

A persistent anomaly is detected in the HRV measurement, while average heart rate, activity and respiration are determined to be nominally normal for low to moderate activity. Specifically, HRV (e.g., SDNN, RMSSD, SDSD, SDANN) is indicated to be elevated. In view of the known propensity for cardiac arrhythmia, an abnormal and persistent elevated HRV indicates potential onset. The subject is provided with a ‘detect’ signal—a signal that advises the cessation of current activity and assuming a resting state.

Continued monitoring of HRV continues to indicate the abnormal condition. The device can switch to an intensive analysis (a multi-tiered approach to detection and analysis) of the ECG signal (or, depending upon the embodiment, conduct parallel analysis) specifically assessing for P-wave conditions, QRS interval deviations and Q-T interval variance. Such analysis can be accomplished real time, and, should such further analysis indicate anomalous conditions consistent with CA, the subject can receive a second real time notification—indicating a need to seek further medical attention.

In other instances, average heart rate is elevated (tachycardia) or depressed (bradycardia), potentially with a lowering of HRV (e.g., SDNN, RMSSD, SDSD, ApEn, SampEn, FuzzEn, FuzzMEn). In either case, when combined with patient propensity, activity and respiration, each condition can be detected as an onset of cardiac arrhythmia. The subject is provided with a ‘detect’ signal—a signal that advises the cessation of current activity and assuming a resting state. Again, utilizing a parallel or multi-tiered approach, the ECG can be analyzed in real time, and, should such further analysis indicate anomalous conditions consistent with CA, the subject can receive a second real time notification—indicating a need to seek further medical attention.

In the need to detect and action false negatives and false positive responses (as the greatest impact on efficacy), this multiple tier approach to detection, analysis and notification provides for mitigation of potential noise or misleading analysis by minimizing spurious input from motion or unusual activity. It further facilitates the progressive awareness of the subject to a potential condition, providing incremental notification and interaction to facilitate proper action on the part of the subject. Such a multiple tier approach also facilitates the reduction of false positive analysis as non-consequential variability is reduced by the subject. It is further possible to utilize standard thresholds, dynamic thresholds and/or user specific thresholds at each tier, refining the detection and analysis to be accurate, user specific and real-time responsive.

It is notable to recognize that in utilizing this multiple tier approach to detection and analysis, the device is capable of changing modes of operation including sensitivity, resolution, acquisition rate, sampling and other characteristics that can positively impact detection and analysis. This is an aspect of this invention.

In monitoring HRV, a person with diabetes (diabetes mellitus) can identify changes in their systemic condition potentially indicating the onset of a diabetic episode. First order monitoring (e.g., continuous BGM) is an emerging capability, however, is expensive and only used for the most intensive care protocols.

A person with diabetes (the subject) utilizes the invention herein described to monitor, analyze and receive a real time alert in the event of a lowered HRV (e.g., SDNN, SDANN, RMSSD, pNN50, HF, LF, LF/HF). A systemic, gradual decrease in HRV can indicate a deteriorating condition over time, while a sudden decrease (inconsistent with other factors such as heart rate, respiration, and activity) is potentially indicative of the onset of a diabetic episode.

When such a sudden decrease is identified, further monitoring is called for—e.g., for determining hyperglycemia or hypoglycemia. A real time notification, for the subject and for the subject's care team, as said condition is detected, provides the opportunity to immediately deliver corrective action to control blood sugar levels. Through real time monitoring, a substantial diabetic episode can be averted.

In the case of long term HRV reduction, while diabetic autonomic neuropathy rarely causes severe symptoms, it has been identified as strongly associated with a substantial mortality risk. Specifically, cardiac autonomic neuropathy, frequently detected as a reduction in HRV, has been associated with increased mortality in diabetes, cardiovascular disease, and aging. Autonomic imbalances affecting sympathetic and parasympathetic nervous system function may be the strongest predictors of poor cardiovascular outcome and the risk of sudden death. With diabetes as a primary contributor to cardiovascular disease, the relationship of HRV, diabetes and cardiovascular health and the monitoring of HRV is indicative of a maintaining or a deteriorating condition.

Severe allergies affect millions of individuals and can cause a severe drop in blood pressure, cardiac arrest or anaphylaxis. Such reactions can be caused by common food allergies (e.g., peanut) with only trace amounts of allergens causing fatal and near fatal reactions.

A person monitoring HRV (e.g., SDNN, RMSSD, pNNx) when an allergic reaction is encountered may experience a lowering of HRV and an increase in heart rate, unassociated with, for example, increased physical activity.

A child, with a peanut allergy, attending school may inadvertently contact a food allergen. Through such monitoring and analysis, early, real time notification of impending reactions are provided. Real time notification in a system providing both local (i.e., subject) notification and connected alerting (e.g., via cloud connectivity) provides alerts to the child (directly), their instructor, school personnel and parents. Such a system allows for decision and treatment time to be managed effectively. It is often necessary to administer treatment immediately; such monitoring provides early detection, analysis and real time notification. With such notification, treatment can be effectively administered by qualified personnel.

A person in a stress situation, monitoring HRV, may show similar monitoring profiles to an allergic reaction. It is of consequence to utilize prior knowledge of a person's allergic susceptibility (e.g., peanut, tree nut) and situational conditions (e.g., work stress) to identify causal HRV effects and to take appropriate action. Early real time notification of HRV depression combined with other parameters allows for the effective addressing of potentially critical situations.

The determination of metabolic response (blood lactate) and autonomic response (HRV) provides for the real time detection and analysis of the anaerobic threshold (AT) during physical exertion. Such a determination is useful for athletes (training and competition) and for patients (e.g., cardiac rehabilitation).

A subject, using the herein described invention, detects, analyzes and receives real time notification of the crossing of the anaerobic threshold while under exertion. The device, monitoring HRV (e.g., SDNN, SD1) in combination with heart rate and activity, analyzes the HRV level against pre-determined, dynamically determined and/or user specific limits and notifies the user that AT has been crossed.

For dedicated athletes, HRV can be used to train to achieve higher VO2max levels and, during competition, to maximize cardiovascular performance. For a recovering cardiac patient, the herein described invention can provide for therapy at the correct level to optimize recuperation. One advantage of the invention is that in providing direct, real time feedback to the user, it directly impacts the user's desired behavior by providing detection, analysis and feedback for their physiological state.

Monitoring epilepsy via HRV potentially provides an opportunity to forewarn the user of an impending seizure. Real time monitoring enables a user to prevent injury resulting from the surprise onset of seizure.

A user receives real time notification of a potential impending episode by monitoring HRV (SDNN, LF, HF, LF/HF, SD1, SD2, SD1/SD2) with heart rate and activity. By characterizing and maintaining user specific limits, detection and analysis can utilize standard thresholds, dynamic thresholds and/or user specific thresholds for real time notification. By employing a multiple tier approach, a user can be advised to reduce risk of injury (e.g., from a fall) while continued monitoring and analysis continues.

HRV, heart rate and activity as co-parameters for the detection, analysis and notification of chronic pain, effective mitigation and mis-use of pain medication is being studied. There is a direct, substantial correlation of pain to HRV (HF, LF, pNNxx, LF/HF) and it is indicated (literature) that HRV can be a marker for chronic pain. Opioid use for pain alleviation correlates with changes in HRV. In the absence of pain, the changes and correlation has potentially different characteristics.

A patient (e.g., chronic pain sufferer) monitors HRV across varying conditions (e.g., un-treated, under analgesic therapy). The described device builds a user profile for varying conditions. Correlation to these conditions (and associated therapy) would be used to support optimized pain management and mitigate therapy mis-use. Real time notification, combined with care team notification and record keeping supports effective therapy.

In keeping with the need to substantially reduce the impact of false negative and false positive notifications to fulfill safety and efficacy requirements, the approach of multiple tier detection, analysis and notification is vital. The device, as described herein, is capable of changing data collection, filtering, analysis and the approach to parameter inclusion, as well as the method, means and type of real time notification. Further, as a connected, albeit self-contained device, there is further mitigation from failure due to external communication, interference and system reliability, while maintaining the ability to incorporate a broader system (e.g., cloud based, external device—smart phone based) architecture.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although as described in the preferred embodiment of the present invention, it will be readily apparent to those skilled in the art that modifications may be made thereto which do not exceed the scope of the appended claims. Therefore, the scope of the invention is only to be limited by the following claims. Reference numbers recited herein are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A method for real-time detection, analysis, and application of heart rate variability (HRV) using a wearable device, the method comprising:
(a) detecting a plurality of consecutive heartbeat related waves and recording a plurality of consecutive time values for each wave (200, 201, 202, 203);
(b) determining and recording a plurality of consecutive of time intervals between the time values to generate a temporal interval array (204);
(c) analyzing the plurality of consecutive heart beat related waves, time values, and time intervals for suitability of use for HRV related applications (207);
(d) detecting ectopic beats and correcting the time values and temporal intervals in the temporal interval array associated with the ectopic beats (205, 206);
(e) computing one or more HRV measurements from the plurality of consecutive temporal intervals in the temporal interval array (208);
(f) analyzing said HRV measurements against one or more user established thresholds to determine one or more HRV measurement excursions from said established user limit(s) (209);
wherein the one or more HRV measurements and the one or more HRV measurement excursions are combined with one or more additional predetermined or dynamically set inputs (210, 211) to determine when an alert is sent to one or more pre-determined parties via a real-time notification (212).

2. The method of claim 1, wherein the one or more user established limits (211) comprises upper limits, lower limits or variability limits.

3. The method of claim 1, wherein the one or more user established limits (211) comprises predetermined limits or dynamically determined limits.

4. The method of claim 1, wherein the one or more additional predetermined or dynamically set inputs (212) are user inputs, application specific inputs or dynamically determined inputs.

5. The method of claim 1, wherein the method of generating one or more HRV measurements from the based on data in the temporal interval comprises one or more of: standard deviation of normal to normal R-R intervals (“SDNN”), root mean square of successive N-N interval differences (“RMSSD”), standard deviation of successive differences (“SDSD”), standard deviation of averages of N-N intervals (“SDANN”), triangular interpolation of N-N interval histogram (“TINN”), NN count divided by total NN interval (“pNNx”), Hilbert Transform, approximate entropy (“ApEn”), sample entropy (“SampEn”), fuzzy entropy (“FuzzEn”), fuzzy measure entropy (“FuzzMEn”), or spectral HRV measurements including: power in very low frequency (“VLF”), power in low frequency (“LF”), power in high frequency (“HF”), a ratio of LF to HF, standard deviation of Poincare plot perpendicular to a line of identity (“SD1”), standard deviation of axis of Poincare plot parallel to the line of identity (“SD2”), or a ratio of SD1 to SD2.

6. The method of claim 1, wherein the one or more predetermined or dynamically set inputs comprise: respiration rate, blood pressure value, body temperature, level of physical motion, heart rate, cardiac arrhythmia, a pre-determined condition marker, a pre-existing condition specification, or a level of electrodermal activity.

7. The method of claim 6, wherein the one or more pre-determined or dynamically set inputs includes one or more figures of merit for cardiac arrhythmia, including at least one of rapid heart rate, slowed heart rate, irregular heart rate, long QT syndrome, or fibrillation, when such an input is analyzed as deviating from an established baseline.

8. The method of claim 7, wherein the said one or more figures of merit for cardiac arrhythmia is related to PQRST wave analysis for heart rate, p-wave, P-R interval, QRS complex interval, ST segment elevation or depression, or QTc interval.

9. The method of claim 1, wherein the one or more predetermined parties comprises: the user, the user's care team, a medical professional, or a medical provider.

10. The method of claim 1, wherein the real-time notification comprises one or more of: a haptic indicator, an auditory indicator, or a visual indicator.

12. A wearable device (101) for real-time detection, analysis, and application of heart rate variability (HRV), the device comprising:
(a) a chest strap (120) or wearable article integrated with one or more strap electrodes to detect a plurality of consecutive PQRST waves; and
(b) a battery-powered, self-contained processing circuit (110) with a microprocessor, an electrocardiogram (“ECG”) analog front end, an onboard parameter set capability (305), a non-volatile memory (308); and
(c) computer readable instructions executable by said microprocessor (301) causing the microprocessor (301) to:
(i) detect a plurality of consecutive heartbeat related waves and recording a time value for each wave;
(ii) determine and record an interval for each time value pair to generate a temporal interval array;
(iii) analyze the plurality of consecutive heart beat related waves, each time value, and each interval for suitability of use for HRV related applications;
(iv) correct each time value and temporal interval associated with ectopic beats;
(v) determine one or more HRV measurements from the based on data in the temporal interval array;
(vi) analyze said HRV measurements against one or more user established limits to determine one or more HRV measurement excursions from said established user limit(s);
wherein the one or more HRV measurements and the one or more HRV measurement excursions are combined with one or more additional predetermined or dynamically set inputs to determine when an alert is sent to one or more pre-determined parties via a real-time notification.

13. The device of claim 12, wherein the one or more user established limits comprises upper limits, lower limits or variability limits.

14. The device of claim 12, wherein the one or more user established limits comprises predetermined limits or dynamically determined limits.

15. The device of claim 12, wherein the one or more additional predetermined or dynamically set inputs are user inputs, application specific inputs or dynamically determined inputs.

16. The device of claim 12, wherein the method of generating one or more HRV measurements from the based on data in the temporal interval comprises one or more of: standard deviation of normal to normal R-R intervals (“SDNN”), root mean square of successive N-N interval differences (“RMSSD”), standard deviation of successive differences (“SDSD”), standard deviation of averages of N-N intervals (“SDANN”), triangular interpolation of N-N interval histogram (“TINN”), NN count divided by total NN interval (“pNNx”), Hilbert Transform, approximate entropy (“ApEn”), sample entropy (“SampEn”), fuzzy entropy (“FuzzEn”), fuzzy measure entropy (“FuzzMEn”), or spectral HRV measurements including: power in very low frequency (“VLF”), power in low frequency (“LF”), power in high frequency (“HF”), a ratio of LF to HF, standard deviation of Poincare plot perpendicular to a line of identity (“SD1”), standard deviation of axis of Poincare plot parallel to the line of identity (“SD2”), or a ratio of SD1 to SD2.

17. The device of claim 12, wherein the one or more predetermined or dynamically set inputs comprise: respiration rate, blood pressure value, body temperature, level of physical motion, heart rate, cardiac arrhythmia, a pre-determined condition marker, a pre-existing condition specification, or a level of electrodermal activity.

18. The device of claim 17, wherein the one or more pre-determined or dynamically set inputs includes one or more figures of merit for cardiac arrhythmia, including at least one of rapid heart rate, slowed heart rate, irregular heart rate, long QT syndrome, or fibrillation, when such an input is analyzed as deviating from an established baseline.

19. The device of claim 18, wherein the said one or more figures of merit for cardiac arrhythmia is related to PQRST wave analysis for heart rate, p-wave, P-R interval, QRS complex interval, ST segment elevation or depression, or QTc interval.

20. The device of claim 12, wherein the one or more predetermined parties comprises: the user, the user's care team, a medical professional, or a medical provider.

21. The device of claim 12, wherein the real-time notification comprises one or more of: a haptic indicator, an auditory indicator, or a visual indicator.

22. A system for real-time detection, analysis, and application of heart rate variability (HRV), the device comprising:
a. The wearable device of claim 12, and
b. A communications means to send and/or receive data to one or more external devices (611), and
c. a real-time notification to the user via said one or more external devices (611).

23. The system of claim 22 with communications (615, 616) to transfer data to one or more of the Internet cloud, a centralized data store, or an analytical data service.

24. The system of claim 23 with communications (617) to transfer data to one or more external devices (613) with said devices (613) capable of delivering notifications or information to one or more of a user, a care team, a medical professional or a medical provider.

25. A method of providing real time notification to a user for monitoring events, conditions or episodes, wherein this real time feedback can be in multiple tiers:
a. a first tier indicating initial detection, analysis and identification of a potential impending event, episode or condition based upon the user's real time behavior
b. a subsequent tier or tiers of notification indicating detection, analysis and identification of a potential impending event, episode or condition based upon the user's modified real time behavior—said modified behavior initiated by training, informing or specifying such modifications as a result of the first tier detection, analysis and identification of a potential impending event, episode or condition and/or modified detection, and/or analysis and and/or identification techniques.
